(19) United States
(12) Patent Application Publication
(10) Pub. No.: US 2008/0233561 A1
(43) Pub. Date: Sep. 25, 2008

(54) Method for Measuring Cytopathic Effect Due to Viral Infection in Cells Using Electric Cell-Substrate Impedance Sensing

(75) Inventor: Eugenia Wang, Prospect, KY (US)

Correspondence Address: STITES & HARBISON, PLLC, 400 W MARKET ST, SUITE 1800, LOUISVILLE, KY, 40202-3352, US

(73) Assignee: UNIVERSITY OF LOUISVILLE RESEARCH FOUNDATION, INC., Louisville, KY (US)

(21) Appl. No.: 11/996,892

(22) PCT Filed: Jul. 19, 2006
(86) PCT No.: PCT/US06/27910

§ 371 Date: Jan. 25, 2008

Related U.S. Application Data
(60) Provisional application No. 60/700,925, filed on Jul. 20, 2005.

(51) Int. Cl. C12Q 1/70

(52) U.S. Cl. 435/5

(57) ABSTRACT

A method of measuring cytopathic effect in cells includes providing cells in culture, using electric cell-substrate impedance sensing (ECIS) to measure the resistance of current associated with the cells, and quantifying the cytopathic effect (CPE) associated with the cells based on the measured resistance. The cells may be identified as being infected with a virus if the CPE associated with the cells is above a predetermined level. Alternatively, the cells may be provided in a healthy monolayer and infected with a virus in order to measure the effect of the virus on CPE associated with the cells. Cells may also be treated with candidate antiviral agents and the effects of the agents on the virus-infected cells may be measured to screen for and identify actual antiviral agents.

### CROSS REFERENCES TO RELATED APPLICATIONS

This application claims priority from U.S. Provisional Application Ser. No. 60/700,925 filed Jul. 20, 2005, the entire disclosure of which is incorporated herein by this reference.

### STATEMENT REGARDING U.S. FEDERALLY SPONSORED RESEARCH

This invention was made with the support of the United States government from Grant Number DAAD19-01-1-04501 awarded by the Defense Advance Research Project Agency. The U.S. government has rights to this invention.

### FIELD OF THE INVENTION

The present invention relates to methods for studying viral infections, screening for viral infections, and screening methods for antiviral agents.

### BACKGROUND OF THE INVENTION

Viruses are responsible for a variety of health problems that can be severe, life threatening and even fatal. For example, according to the World Health Organization (WHO), influenza A virus is thought to be the cause of about 500,000 deaths globally each year (WHO, 2004). Influenza A virus contains a segmented, negative sense, single stranded RNA genome, which is transcribed to mRNA and translated to proteins by an infected cell's enzymes and internal machinery. Avian influenza, sometimes referred to as “bird flu,” is an infectious disease caused by type A strains of the influenza virus.

Although avian influenza viruses do not typically infect humans, a particularly virulent avian influenza virus was introduced into the human population of Hong Kong in 1997, causing severe respiratory disease in those infected and ultimately killing several people. See Claas, et al. Vaccine 16:997-978 (1998); Subbarao, et al. Science 279, 393-396 (1998), which are incorporated herein by this reference. In 2003, another outbreak of avian influenza in Hong Kong resulted in the death of an infected person, an outbreak in the Netherlands caused illness in many and resulted in a death, and three cases of avian influenza in Viet Nam each resulted in death. In 2004, avian influenza virus was found in infected people suffering from severe respiratory disease in Viet Nam. That year there were 29 cases of human infection in Viet Nam, 20 of which resulted in death. Also in 2004, 17 cases of human infections were confirmed in Thailand, 12 of which resulted in death. In 2005, the number of reported cases of avian influenza and the number of countries in which they were reported increased. A total of 95 cases of human infection were confirmed in Cambodia, China, Indonesia, Thailand, and Viet Nam, 41 of which were fatal. During the first half of 2006, the number of countries in which cases were reported of avian influenza in humans again increased, relative to the previous year. A total of 102 cases of human infection were confirmed in Azerbaijan, Cambodia, China, Djibouti, Egypt, Indonesia, Iraq, and Turkey, 66 of which were fatal. (See WHO—Avian influenza “bird flu”—Fact sheet, February, 2006; Weekly Epidemiological Record, Epidemiology of WHO-confirmed human cases of avian influenza A (H5N1) infection, Jun. 30, 2006; Cumulative Number of Confirmed Human Cases of Avian Influenza A/(H5N1) Reported to WHO, Jul. 4, 2006; and Avian influenza—situation in Indonesia—update 21, Jul. 4, 2006).

The initial documented introduction of the virus into the human population in Hong Kong, compounded with the continued circulation of similar viruses in the area lead many to believe a pandemic influenza will occur in the not too distant future. See I(aye, et al. Clin Infect Dis 40, 108-112 (2005); Palese, Nat Med 10, S82-S87 (2004), which are incorporated herein by this reference. With conditions as they are, effective antiviral drugs are needed urgently.

There are currently a variety of methods used to screen drugs for potential antiviral activity. Generally, such screening methods are practiced by infecting healthy cultured cells with a virus of interest and quantitating viable cells. Assays for cell viability or apoptosis may involve a variety of colorimetric, fluorometric or other detection and identification methods. See Smee, et al. J Virol Methods 106, 71-79 (2002), which is incorporated herein by this reference. Common methods of assaying for cell viability include the use of dyes or stains, such as MTT, XTT or TUNEL. Such assays require harvesting cells at particular time points and provide mere “snap shots” of cell number for particular moments-in-time.

In an MTT cell viability assay, for example, MTT is added to a test plate of cells and is reduced in the presence of cells with functioning mitochondria, resulting in a detectable color change. The cells are harvested and the amount of MTT conversion is quantified spectrophotometrically and correlated to cell number based on a series of standards. As such, cell viability is quantitated for the moment-in-time when the cells are harvested. When performing such an assay, data is collected at multiple, generally arbitrary, time points; each time point includes replicates; and samples used to form a series of standards, i.e., standard curve, are extracted at each time point. As such, the collectable data is limited to the number of replicates and standard samples feasible for a given experiment. Additionally, the data is associated only with the arbitrary time points, ignoring the events taking place between time points.

Plaque-forming assays may also be used to study the effects of viral infection on cell viability and have the benefit of being a direct analysis of the cells being tested; however, such methods involve human assessment of cells making them tedious, subjective and prone to human exhaustion and error. Furthermore, such methods again involve the generally arbitrary selection of time points to assess, rather than providing a method for the continuous real-time collection of data.

Other known methods of studying the effects of viral infections and candidate antiviral agents, such as time lapse microscopy, permit the observation of the effects of the viral infection in cell culture in real time, but lack or have particularly limited powers of quantitation.

Accordingly, there remains a need in the art for a method of studying the effects of viral inventions, screening for effective antiviral agents, and screening samples for the presence of viral infections, which satisfactorily addresses the above-identified problems.

### SUMMARY OF THE INVENTION

The present invention addresses the above identified problems by providing a method of measuring cytopathic effect (CPE) in cells using electric cell-substrate impedance sensing (ECIS), which method is useful for studying the effects of viral inventions, screening for effective antiviral agents, evaluating antiviral vaccines, and screening samples for the presence of viral infections. The method of the present invention is objective, automated, allows for a high-throughput of samples, and allows for continuous real-time collection of data.

ECIS is a technology that is not only capable of producing quantitative data, but is also able to monitor experiments in real-time. Because data may be continuously collected from a single group of cells throughout the course of an experiment, rather than at multiple discrete time-points, replicates that are often necessary for other cell viability or apoptosis assays are not necessary and the possible introduction of variability between replicates is effectively eliminated.

Equipment for automated cell monitoring using ECIS may be obtained from Applied BioPhysics, Inc., Troy, N.Y. Although ECIS technology has been used to study cell morphology, cell substrate interactions, cell layer barrier function, cell motility, and wound healing, the use of ECIS technology has not heretofore been contemplated or suggested for measuring cytopathic effect and/or for studying viral inventions and treatments therefore.

ECIS operates in the following manner in the context of the present invention. Cells are grown on the insulated surface of culture dishes or wells of a multi-well culture dish, each dish or well having an electrode lining. Each well includes a plurality of non-insulated circular areas for current flow. A device measures a noninvasive AC current as it flows through culture medium surrounding the cells. As cells attach and spread on the surface of the electrode lining, the current is impeded. A greater number of cells attaching and spreading on the electrode lining will result in and correlate to a greater resistance of current.

Cytopathic effect (CPE) is the degenerative change in cells associated with the multiplication of a virus. CPE due to viral infection is typically characterized by a rounded cell morphology and detachment of cells from the surface of a culture dish. The CPE due to a viral infection results in the release of cells from the surface of the electrode lining, which restores current. Thus, CPE is correlated with the decreased ability of the cell monolayer to impede a noninvasive AC signal—the lesser the resistance, the greater the CPE.

The present invention includes a method of measuring cytopathic effect due to a viral infection in a sample, e.g., cells, using ECIS to quantify the level of CPE by measuring resistance of current in a given sample. Continuous and real-time data indicating the presence of and relating to the effects of viral infections can be collected by this method. The continuous and real-time data can give insight into the rate, progression, and severity of CPE in a given sample. By obtaining information about CPE in a given sample or group of samples, the effects of viral infections may be studied, agents may be screened for anti-viral activity, vaccines for prevention and treatment of viruses may be evaluated, and the presence of a viral infection may be confirmed or denied. The method may be used to measure, quantify and compare CPE associated with cells due to the exposure to: different viruses, different concentrations of viruses, different samples, different candidate antiviral agents, different concentrations of candidate antiviral agents, different candidate vaccines, or different concentrations of candidate vaccines. Additionally, the method may be used to measure, quantify and compare CPE associated with different cell types due to the exposure to: a virus, a sample, a candidate antiviral agent, or a candidate antiviral vaccine.

An exemplary method of the present invention includes: providing cells in culture; using ECIS to measure the resistance of current associated with the cells; and quantifying the cytopathic effect associated with the cells based on the measured resistance. Another exemplary method of the present invention includes: providing cells in culture; using ECIS to measure the resistance of current associated with the cells; and correlating the measured resistance of current associated with the cells. Another exemplary method of the present invention includes: providing cells in culture; using ECIS to measure the resistance of current associated with the cells; and correlating the change in resistance of current over time to apoptotic rate.

### DESCRIPTION OF THE DRAWINGS

FIG. 1A depicts a multi-well culture plate that may be used to collect data using electric cell-substrate impedance sensing (ECIS) and further provides an expanded view of one of the wells, illustrating multiple non-insulated circular areas for current flow;

FIG. 1B provides a cross-sectional view of a non-insulated circular area exposing an electrode lining, and the surrounding insulated surface, of a well of the plate depicted in FIG. 1A;

FIG. 1C provides the cross-sectional view of FIG. 1B and illustrates the impedance of current resulting from cells attaching and spreading on the surface of the electrode lining;

FIG. 1D provides the cross-sectional view of FIG. 1B and illustrates the restoration of current resulting from the cells becoming rounded and detaching from the surface of the electrode lining due to the cytopathic effect caused by viral infection in the cells;

FIG. 2 is a flow chart illustrating the steps involved in an exemplary method of the present invention for quantifying CPE due to an infection by a virus of interest;

FIG. 3 is a flow chart illustrating the steps involved in another exemplary method of the present infection for quantifying CPE due to an infection by a virus of interest;

FIG. 4 is a flow chart illustrating the steps involved in an exemplary method of the present invention for screening for antiviral agents;

FIG. 5 is a flow chart illustrating the steps involved in another exemplary method of the present invention for screening for antiviral agents;

FIG. 6A is a graph depicting resistance as a function of time in uninfected and virus-infected cells at MOIs of 1, 5, and 10;

FIG. 6B includes photographs of the uninfected and virus-infected cells at MOIs of 1, 5, and 10;

FIG. 7A is a graph depicting resistance as a function of time in uninfected cells, cells treated with antiviral agent, virus-infected cells, and virus-infected cells treated with antiviral agent; and

FIG. 7B includes photographs of the uninfected cells, cells treated with antiviral agent, virus-infected cells, and virus-infected cells treated with antiviral agent.

### DESCRIPTION OF EMBODIMENTS OF THE INVENTION

The present invention includes methods for measuring cytopathic effect in cells using electric cell-substrate impedance sensing (ECIS), which are useful for studying the effects of viral inventions, screening for effective antiviral agents, evaluating antiviral vaccines, and screening samples for the presence of viral infections.

ECIS is a technology that is not only capable of producing quantitative data, but is also able to monitor experiments in real-time. See Giaever, et al. Proc Natl Acad Sci USA 81, 3761-3764 (1984), which is incorporated herein by this reference. Because data may be continuously collected from a single group of cells throughout the course of an experiment, rather than at multiple discrete time-points, replicates that are often necessary for other cell viability or apoptosis assays are not necessary and the possible introduction of variability between replicates is effectively eliminated.

Although ECIS technology has been used to study cell morphology, cell substrate interactions, cell layer barrier function, cell motility, and wound healing, the use of ECIS technology has not heretofore been contemplated or suggested for measuring cytopathic effect and studying viral inventions and treatments therefore. See Giaever, et al. Nature 366, 591-592 (1993); Giaever, et al. Proc Natl Acad Sci USA 88, 7896-7900 (1991); Giaever, et al. IEEE Trans Biomed Eng 33, 242-247 (1986); Keese, et al. Proc Natl Acad Sci USA 101, 1554-1559 (2004); Lo, et al. Exp Cell Res 204, 102-109 1993; Mitra, et al. Biotechniques 11, 504-510 (1991); Tiruppathi, et al. Proc Natl Acad Sci USA 89, 7919-7923 (1992); Wegener, et al. “Electric cell-substrate impedance sensing (ECIS) as a noninvasive means to monitor the kinetics of cell spreading to artificial surfaces,” 259, 158-166 (2000), which are incorporated herein by this reference. Equipment for automated cell monitoring using ECIS may be obtained from Applied BioPhysics, Inc., Troy, N.Y.

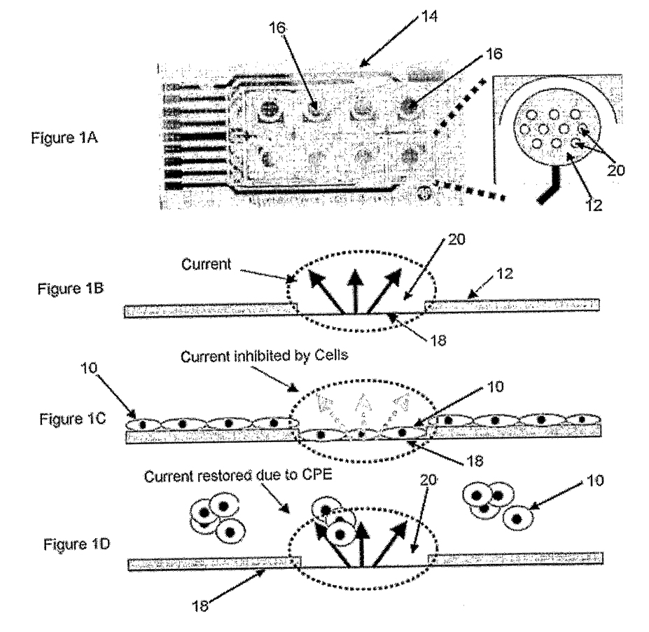

With reference to FIGS. 1A-1D, ECIS operates in the following manner in the context of the present invention. Cells 10 may be grown on the insulated surface 12 of multi-well culture dishes 14, each well 16 having an identical electrode lining 18. Each well 16 includes a plurality of non-insulated circular areas 20 for current flow. A device (not shown) measures a noninvasive AC current as it flows through culture medium surrounding the cells 10. With reference to FIG. 1C, as cells 10 attach and spread on the surface of the electrode lining 18, the current is impeded. A greater number of cells 10 attaching and spreading on the electrode lining 18 will result in and correlate to a greater resistance of current. This signal resistance is accomplished not only through the insulation of the electrode lining 18 by the cell 10 membranes, but also through the tight junctions formed between neighboring cells 10 and the distance the cells 10 are from the substrate to which they are attached.

Cytopathic effect (CPE) is the degenerative change in cells associated with the multiplication of a virus. CPE due to viral infection is typically characterized by a rounded cell morphology and detachment of cells from the surface of a culture dish. With reference to FIG. 1D, the CPE due to a viral infection results in the release of cells 10 from the surface of the electrode lining 18, which restores current. Thus, CPE is correlated with the decreased ability of the cell monolayer to impede a noninvasive AC signal—the lesser the resistance, the greater the CPE. The present invention includes methods of measuring cytopathic effect due to a viral infection in a sample, e.g., cells. These methods use ECIS to quantify the level of CPE by measuring resistance of current in a given sample. Continuous and real-time data indicating the presence of and relating to the effects of viral infections can be collected by this method. The continuous and real-time data can give insight into the rate, progression, and severity of CPE in a given sample. By obtaining information about CPE in a given sample or group of samples, the effects of viral infections may be studied, agents may be screened for anti-viral activity, vaccines for prevention and treatment of viruses may be evaluated, and the presence of a viral infection may be confirmed or denied.

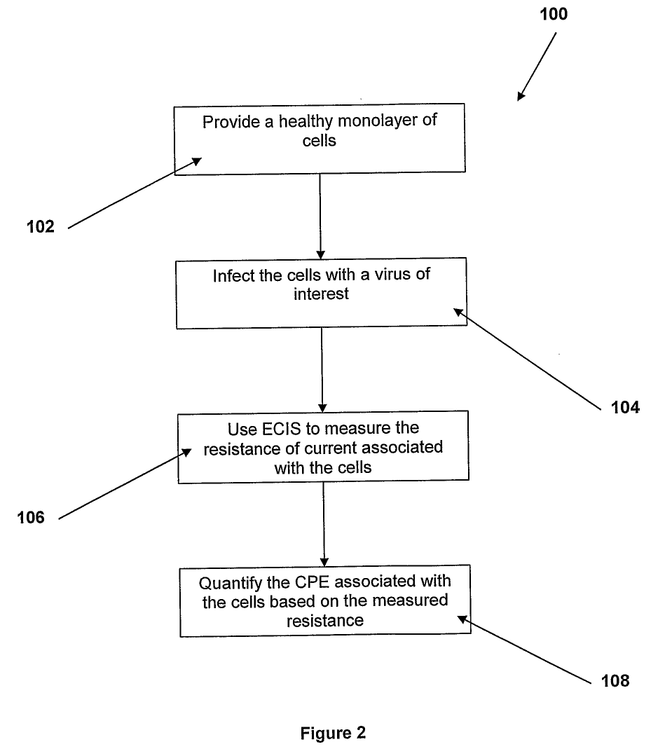

Exemplary methods of measuring CPE due to an infection by a virus of interest will now be described. With reference to FIG. 2, an exemplary method 100 of the present invention includes the following steps: providing a healthy monolayer of cells 102; infecting the cells with the virus of interest 104; using ECIS to measure the resistance of current associated with the cells 106; quantifying the CPE associated with the cells based on the measured resistance 108.

With regard to the step of providing the healthy monolayers of cells 102, one or more culture dishes or multi-well culture plates having electrode linings may be provided for seeding the cells. The cells are seeded at a predetermined concentration in growth medium and the cells are allowed to form a healthy monolayer.

After the cells are seeded, the resistance data begins to be collected. When the resistance reaches a plateau, the cells are infected with the virus of interest 104. The resistance data continues to be collected using ECIS 106 until the occurrence of a predetermined event, for example, the event may be the passing of a predetermined period of time, the measured resistance within a particular plate or well reaching a predetermined level, or another predetermined event.

The CPE may then be quantified based on the measured resistance 108. This quantification step may include comparing the quantified CPE associated with cells following infection with the CPE associated with the cells prior to infection, corrected to account for cell growth occurring during the course of the data collection. Additionally or alternatively, this quantification may include comparing the quantified CPE associated with the cells to a standard curve plotting CPE as a function of resistance. The level of CPE may be assessed for all time points during period that resistance data is being continuously gathered. The rate of change of CPE may be assessed by calculating the change in the level of CPE over a given period of time. The severity of the CPE may be assessed by considering both the level and rate of change of CPE. For example, the CPE may be considered more sever if both the level and rate of change of CPE are relatively high.

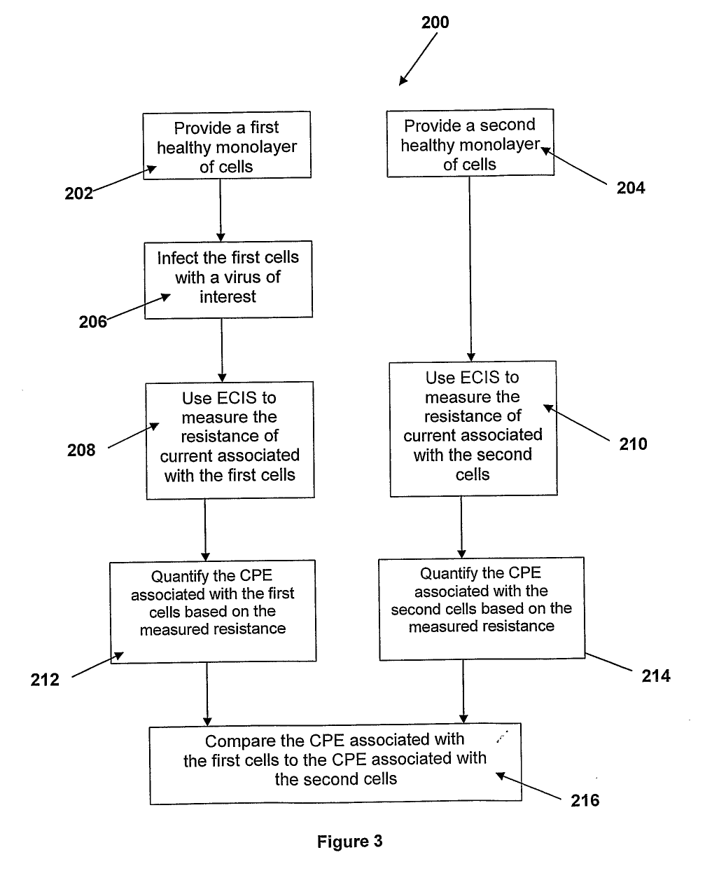

With reference to FIG. 3, another exemplary method 200 of the present invention for quantifying the level, the rate, and/or the severity of CPE due to an infection by a virus of interest includes the following steps: providing a first healthy monolayer of cells 202; providing a second healthy monolayer of cells 204; infecting the first cells with the virus of interest 206; using ECIS to measure the resistance of current associated with the first cells 208; using ECIS to measure the resistance of current associated with the second cells 210; quantifying the CPE associated with the first cells based on the measured resistance 212; quantifying the CPE associated with the second cells based on the measured resistance 212; and comparing the CPE associated with the first cells to the CPE associated with the second cells 216.

With regard to the step of providing first and second healthy monolayers of cells 202, 204, a multi-well culture dish or individual culture dishes having electrode linings may be provided for seeding the cells. At least one well or dish is designated to receive cells that will be infected with the virus of interest (first cells), and at least one well or dish is designated to receive cells that will not be infected with the virus of interest and will serve as a control (second cells). The cells are seeded in the appropriate wells or dishes at a predetermined concentration in growth medium and the cells are allowed to form a healthy monolayer. The cells are seeded in the wells or dishes at the same concentration, such that the second cells may serve as an internal control for cell growth occurring during the course of the data collection.

After the cells are seeded, the resistance data begins to be collected. When the resistance reaches a plateau, the first cells are infected with the virus of interest 206. The resistance data continues to be collected using ECIS 208, 210 until the occurrence of a predetermined event. The resistance associated with the first cells is used to quantify the CPE associated with the first cells 212. Likewise, the CPE associated with the second cells is quantified based on the measured resistance 214. Because the first cells are infected with the virus and the second cells are not, it is expected that the resistance associated with the first cells would be less than the resistance associated with the second cells, and it is therefore expected that the CPE associated with the first cells would be greater than the CPE associated with the second cells. The CPE due to the infection of the first cells by the virus of interest may then be determined by comparing the CPE associated with the first cells to the CPE associated with the second cells 216.

Other exemplary methods of the present invention for measuring CPE due to an infection by a virus of interest may include the use of additional distinct monolayers of cells provided in culture plates or wells. These additional monolayers of cells can be designated to be infected with different concentrations of the virus of interest, wherein resistance data may be gathered to quantify and compare the CPE associated with each distinct monolayer of cells. Other exemplary methods may include the use additional monolayers of cells provided in culture dishes or wells, which additional monolayers of cells are of a different cell types, such that the CPE associated with the different cell types may be compared, allowing differences in the effect of a virus on different cell types to be determined. For example, the effect of a virus on human cells could be compared to the effect of the virus on mouse cells. As a refinement to the exemplary methods, further culture dishes or wells may be designated to serve as additional controls, which may receive, for example, growth medium containing no cells or growth medium containing the virus of interest and no cells.

Exemplary methods of the present invention for screening for antiviral agents will now be described. Antiviral agents have the ability to reverse CPE, resulting in a renewed monolayer of cells adhering to the surface of a culture dish, which results in increased resistance in the current—the greater the resistance, the lesser the CPE. Continuous and real-time data related to the effects of a candidate antiviral agent on a viral infection can be collected by this method. The continuous and real-time data can give insight into the efficacy and efficiency of the antiviral agent against the viral infection.

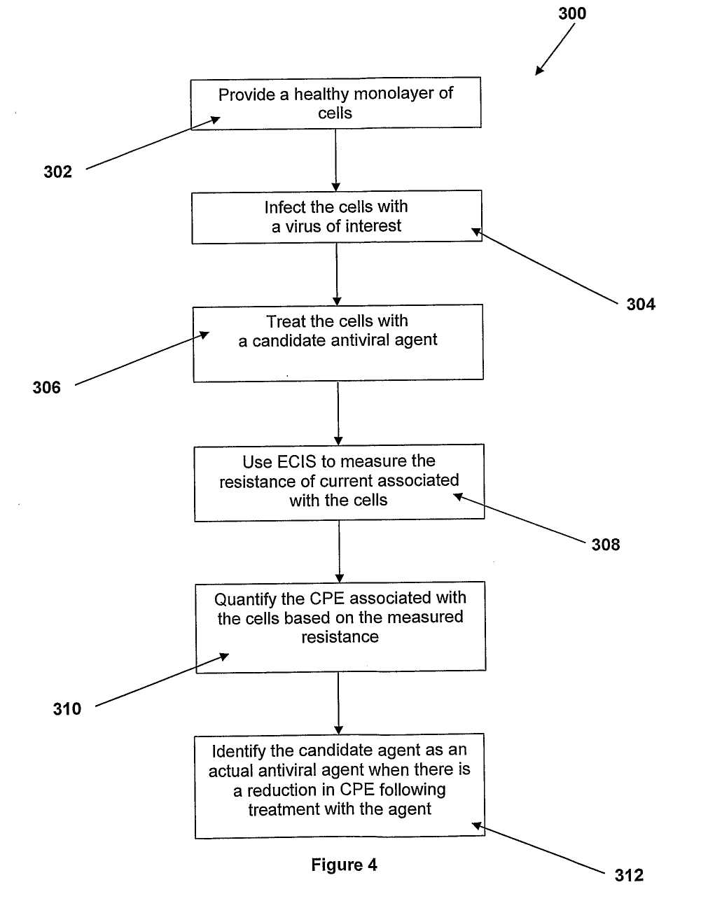

With reference to FIG. 4, an exemplary method 300 of the present invention for screening for antiviral agents includes the following steps: providing a healthy monolayer of cells 302; infecting the cells with a virus of interest 304; treating the cells with a candidate antiviral agent 306; using ECIS to measure the resistance of current associated with the cells 308; quantifying the CPE associated with the cells 310; and identifying the candidate agent as an actual antiviral agent when there is a reduction in CPE following treatment with the agent 312.

With regard to the step of providing the healthy monolayer of cells 302, one or more culture dishes or multi-well culture plates having electrode linings may be provided for seeding the cells. The cells are seeded at a predetermined concentration in growth medium and the cells are allowed to form a healthy monolayer.

After the cells are seeded, the resistance data begins to be collected. When the resistance reaches a plateau, the cells are infected with the virus of interest 304. The cells are then treated with a candidate antiviral agent 306. The resistance data continues to be collected using ECIS 308 until the occurrence of a predetermined event (e.g., the passing of a predetermined period of time). If the candidate agent is having an antiviral effect, the resistance will begin to increase, as the agent begins to reverse CPE and a renewed monolayer of cells begins to form on the surface of the culture dish or well. The CPE associated with the cells is then quantified based on the measured resistance 310. The greater the resistance affected by a renewed monolayer of cells, the lesser the CPE associated with the cells. The candidate agent is identified as an actual antiviral agent if there is a reduction in CPE following treatment with the agent 312.

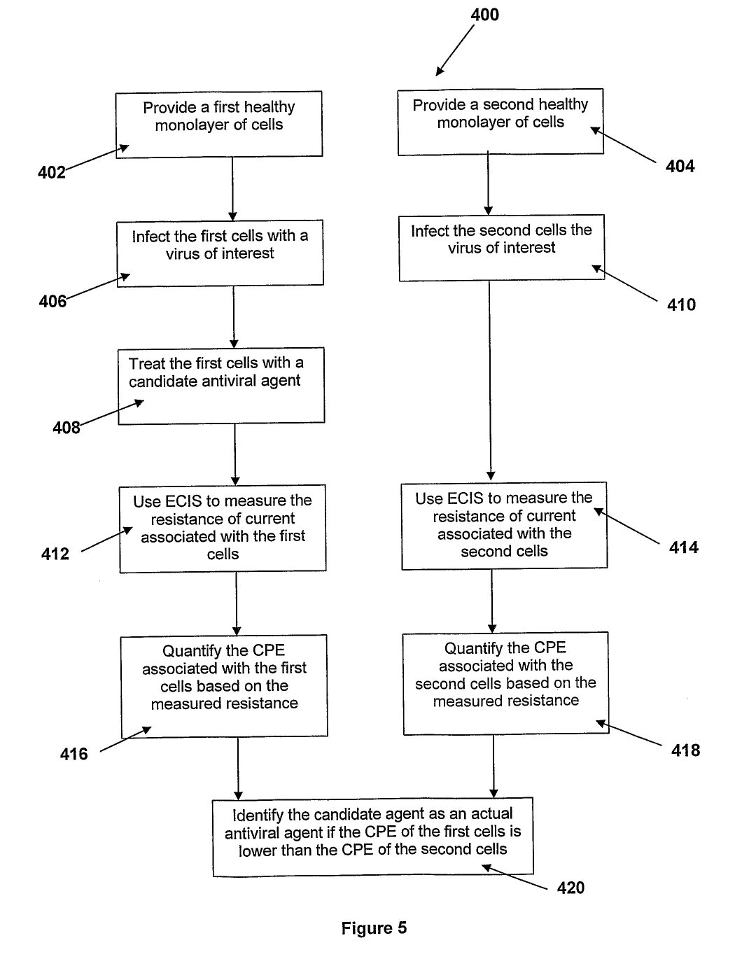

With reference to FIG. 5, another exemplary method 400 of the present invention for screening for effective antiviral agents includes the following steps: providing a first healthy monolayer of cells 402; providing a second healthy monolayer of cells 404; infecting the first cells with a virus of interest 406; infecting the second cells with the virus of interest 408; treating the first cells with a candidate antiviral agent 410; using ECIS to measure the resistance of current associated with the first cells 412; using ECIS to measure the resistance of current associated with the second cells 414; quantifying the CPE associated with the first cells based on the measured resistance 416; quantifying the CPE associated with the second cells based on the measured resistance 418; and identifying the candidate agent as an actual antiviral agent if the CPE of the first cells is lower than the CPE of the second cells 420.

With regard to the step of providing the healthy monolayers of cells 402, 404, one or more culture dishes or multi-well culture plates having electrode linings may be provided for seeding the cells. At least one well or dish is designated to receive cells that will be infected with the virus of interest and will also be treated with the candidate antiviral agent (first cells). At least one well or dish is designated to receive cells that will be infected with the virus of interest and will not be treated with the candidate antiviral agent (second cells). The cells are seeded at a predetermined concentration in growth medium and the cells are allowed to form a healthy monolayer.

After the cells are seeded, the resistance data begins to be collected. When the resistance reaches a plateau, the cells are infected with the virus of interest 406, 408. The first cells are then treated with a candidate antiviral agent 410. The resistance data continues to be collected using ECIS 412, 414 until the occurrence of a predetermined event (e.g., the passing of a predetermined period of time, a predetermined level of resistance is attained within a particular well or plate). If the candidate agent is having an antiviral effect, the resistance associated with the first cells will begin to increase, relative to the resistance associated with the second cells, as the agent begins to reverse CPE and a renewed monolayer of first cells begins to form on the surface of the culture dish or well. The CPE associated with the cells is then quantified based on the measured resistance 416, 418. The greater the resistance affected by a renewed monolayer of cells, the lesser the CPE associated with the cells. The candidate agent is identified as an actual antiviral agent if the CPE of the first cells is lower than the CPE of the second cells 420.

Other exemplary methods of the present invention for screening for antiviral agents include: providing cells that are infected with a virus; treating the cells with a candidate antiviral agent; using ECIS to measure the resistance of current associated with the cells; quantifying the CPE associated with the cells based on the measured resistance; and identifying the candidate agent as an actual antiviral agent when there is a reduction in CPE following treatment with the agent. In this regard, the provided cells may be infected with a identified or an unidentified virus. For example, cells may be obtained from an animal believed to be infected with an unknown virus and the exemplary method may be used to screen for agents having antiviral activity directed towards this unknown virus.

Other exemplary methods of the present invention for screening for antiviral agents may include the use of additional distinct monolayers of cells provided in culture plates or wells. These additional monolayers of cells can be designated to be infected with a concentration of the virus and/or a concentration of the candidate antiviral agent. In certain exemplary methods, the additional monolayers of cells may be infected with different concentrations of the virus of interest, wherein resistance data may be gathered to quantify the CPE and compare the CPE associated with each distinct monolayer of cells. In certain exemplary methods, the additional monolayers of cells may be treated with different concentrations of the candidate antiviral agent, wherein resistance data may be gathered to quantify and compare the CPE associated with the cells receiving different concentrations of the agent. In certain exemplary methods, additional healthy monolayers of cells that are each infected with a concentration of the virus and/or treated with a concentration of the candidate antiviral agent may be provided in culture dishes or wells, which additional monolayers of cells are of different cell types. The CPE associated with the different cell types may then be compared.

Other exemplary methods of the present invention may include the use of additional culture plates of wells that are designated to receive additional monolayers of cells that are neither treated with the virus of interest nor the candidate antiviral agent, which may serve as additional controls, for example, controls used to correct for cell growth occurring during the course of the data collection (third cells). As a refinement to the exemplary methods, further culture dishes or wells may be designated to serve as additional controls, which may receive, for example, growth medium containing no cells, growth medium containing the virus of interest and no cells, or growth medium containing the candidate anti-viral agent and no cells.

Other exemplary methods may include treating the cells with a candidate antiviral agent before treating the cells with a virus of interest, wherein a quantified CPE that is below a predetermined level would identify the candidate agent as an actual antiviral agent. Such an exemplary method is useful to assess the ability of the agent to prevent infection. As another refinement, further culture dishes or wells may be designated to receive additional monolayers of cells of a different cell type, such that the effect of a virus of interest on different cell types may be compared, i.e., the CPE associated with the different cell types may be compared; for example, the effect on human cells could be compared to the effect on mouse cells.

In this regard, the present invention also includes methods for evaluating vaccines. An exemplary method of the present invention for evaluating vaccine effectiveness includes the following steps: providing a healthy monolayer of cells; treating the cells with a candidate vaccine; infecting the cells with a virus of interest; using ECIS to measure the resistance of current associated with the cells; quantifying the CPE associated with the cells based on the measured resistance; and identifying the vaccine as an effective vaccine if the CPE associated with the cells drops below a predetermined level.

Another exemplary method of the present invention for evaluating vaccine effectiveness includes the following steps: providing a first healthy monolayer of cells; providing a second healthy monolayer of cells; treating the first cells with a candidate vaccine; infecting the first and second cells with a virus of interest; using ECIS to measure the resistance of current associated with the first and second cells; quantifying the CPE associated with the first and second cells based on the measured resistance; and identifying the vaccine as an effective vaccine if the CPE associated with the first cells is lower than the CPE associated with the second cells.

The present invention may also be used to identify the presence of a virus in a sample. An exemplary method of the present invention for identifying the presence of a virus in a sample includes the steps of: providing a healthy monolayer of cells; exposing the cells to the sample of interest; using ECIS to measure resistance of current associated with the cells; correlating the measured resistance to the CPE associated with the cells; and identifying the sample as containing a virus if the CPE associated with the cells is above a predetermined level. Examples of samples that may be tested for the presence of a virus include: soil, water, food, or animal tissue samples.

The present invention may also be used to identify the presence of a virus in a cells. An exemplary method of the present invention for identifying the presence of a virus in cells includes the steps of: providing cells in culture; using ECIS to measure the resistance of current associated with the cells; correlating the measured resistance to the cytopathic effect associated with the cells; and identifying the cells as being infected by a virus is the CPE associated with the cells is above a predetermined level. The cells may be obtained, for example, form an animal tissue sample. The animal tissue sample could be human. The animal tissue sample could also be obtained from animals including birds, pigs, and cows. The animal tissue sample could also be obtained from a animal that is used for human consumption.

The presence of a virus and the effect of the virus on CPE may be compared for different samples. For example, an exemplary method of the present invention includes the steps of: providing first cells in a healthy monolayer; providing second cells in a healthy monolayer; exposing the first cells to a concentration of a sample; exposing the second cells to a concentration of a sample; using ECIS to measure the resistance of current associated with the cells; correlating the measured resistance to the CPE associated with the cells; and comparing the CPE associated with the first cells to the CPE associated with the second cells. The samples may be obtained, for example, from the soil, water, food, or animal tissue. The samples may be obtained from different geographical regions or different species in the same geographical region. The viral infections of cross-geographic regions may be compared. For another example, differences in avian flu between North American birds and Asian birds may be assessed. For yet another example, differences between an infection in humans, birds, cows, and pigs may be assessed. For still another example, the first cells may be of a different cell type than the second cells. In this regard, the first cells may be exposed to the same sample as the second cells that the effect of the sample on the different cell types may be compared. For yet another example, the first and second cells may be exposed to different concentrations of the samples, allowing the effects of different concentrations of the samples to be compared.

The present invention may also be used to measure the apoptotic rate of cells in culture. Apoptotic rate of cells is generally associated with a rounding of the cells, and often precipitates in their detachment from the substratum. Thus, apoptotic rate in a culture can be measured by ECIS, wherein a decrease in the resistance of current is associated with an increased apoptotic rate. An exemplary method of the present invention for measuring apoptotic rate of cells includes the steps of: providing cells in culture; using ECIS to measure the resistance of current associated with the cells; and correlating the change in resistance of current over time to apoptotic rate. Other exemplary methods of the present invention additionally includes the step of identifying the cells as being infected with a disease-causing agent or pathogen when the apoptotic rate is above a predetermined level. Other exemplary methods of the present invention include the steps of treating the cells with a candidate treatment agent, and identifying the candidate agent as an effective treatment agent when there is a reduction is apoptotic rate following treatment with the agent. Other exemplary methods of the present invention include the steps of providing cells in a healthy monolayer; insulting the healthy monolayer of cells with a disease-causing agent or pathogen; treating the cells with a candidate treatment agent; and identifying the candidate agent as an effective treatment agent when there is either a reduction in apoptotic rate following treatment with the agent, or the apoptotic rate associated with the cells is below a predetermined level.

The above-described exemplary methods are merely examples of methods that may be performed in accordance with the present invention to quantify CPE or apoptotic effect associated cells and various modification and/or refinements will become apparent to those skilled in the art upon reviewing the present document.

The cells that may be used to practice the methods of the present invention include cells that may be cultured. Examples of cells that may be used include, but are not limited to: kidney cells, including, African Green Monkey Kidney (Vero) cells, MDCK cells, CEK (Chicken embryonic kidney) cells, and rhesus monkey kidney cells; epithelial cell lines, including, mouse and human airway epithelial cell, differentiated hamster tracheal epithelial cells, Mv1Lv cells, Human embryonic lung cells, ZHL16C cells, ear epithelial cells dedifferentiated epithelial cells, Vero E6 cell epithelial cells, and human lung carcinoma (A549); Fibroblast cells, including, human foreskin fibroblasts, and dedifferentiated fibroblasts; and other cells, including, Per. C6 cells, BHK-21 C13 cells, HuH7 cells, BGM cells, A549 cells, MRC-5 cells, PRMK cells, R-mix cells, Hu7 cells, human retinoblastoma cell, and human hepatocytes.

The viruses that may be tested and for which antiviral agents may be screened in accordance with the present invention include, but are not limited to: influenza A virus, influenza B virus, other influenza viruses, parainfluenza viruses, Sendai virus, Sindbis virus, hepatitis B virus, hepatitis C virus, other hepatitis viruses, adenoviruses, rhinoviruses, coronaviruses, poliovirus type 3, coxsackie virus B1, coxsackie B3, and other coxsackie viruses, other enteroviruses, Akabane virus, Aino virus, Chuzan virus, herpes simplex viruses, herpes zoster viruses, yellow fever, measles virus, parvovirus, human cytomegalovirus, Moloney murine leukemia virus, encephalomyocarditis virus, severe acute respiratory syndrome/coronavirus, oncolytic adenovirus, West Nile virus, Japanese encephalitis virus, bovine viral diarrhea viruses, human T-cell leukemia virus type-1, maedi-visna virus, vesicular stomatitis virus, swine flu A, echovirus, cytomegalovirus, rubella virus, respiratory syncytial viruses, and human metapneumoviruses Although it is possible to collect continuous and real-time data using the methods of the present invention, if desired, one may collect data at one or more discrete time points without departing from the sprit and scope of the present invention.

The present invention is further illustrated by the following specific but non-limiting example. The following example is prophetic, notwithstanding the numerical values, results and/or data referred to and contained therein.

### EXAMPLE

Influenza A is chosen as a virus infection of interest and MDCK cells are used to study the virus of interest. The exemplary study described herein indicates that, as the CPE caused by influenza A virus infection becomes more severe, the signal resistance from the cell monolayer is reduced in a dose-dependent manner. Additionally, upon pretreatment with ammonium chloride (NH₄Cl), which is known to inhibit virus entry into a cell, the reduction in signal resistance due to influenza infection is abolished. See Jakeman, et al, J Gen Virol 72, 111-115 (1991), which is incorporated herein by this reference and contains a discussion of the ability of NH₄Cl to inhibit virus entry into a cell. The efficacy of the method of the present invention is illustrated by the exemplary study described herein, which method is useful, for example, in the investigation of processes affecting the rate and severity of CPE in cell culture including, but not limited to, antiviral drugs and signal transduction pathways affecting virus replication.

An 8W10E multi-well culture dish is obtained from Applied BioPhysics, Inc. and equilibrated with 200 μL of growth medium (EMEM) including 10% fetal bovine serum (FBS) and antibiotics) per well for 30 minutes in a humidified, 37° C., 5% CO₂ incubator. Each well is seeded with 1×10⁵ MDCK cells in growth medium. ECIS data collection is initiated upon seeding the cells. Once the resistance has reached a plateau, generally between 24-36 hours post-seeding, the cells are washed twice with serum free EMEM containing antibiotics and subsequently infected in duplicate with influenza/A/PR/8/34 virus. The virus is diluted in serum-free EMEM containing 1 μg/mL TPCK treated trypsin, antibiotics and included MOIs of 1, 5, or 10 in addition to mock controls. Following a one hour inoculation period, the virus is removed and replaced with maintenance medium (EMEM containing 0.125% bovine serum albumin (BSA) and antibiotics). ECIS data are collected continuously at 400, 4,000 and 40,000 Hz frequencies until approximately 48 hours post infection (PI).

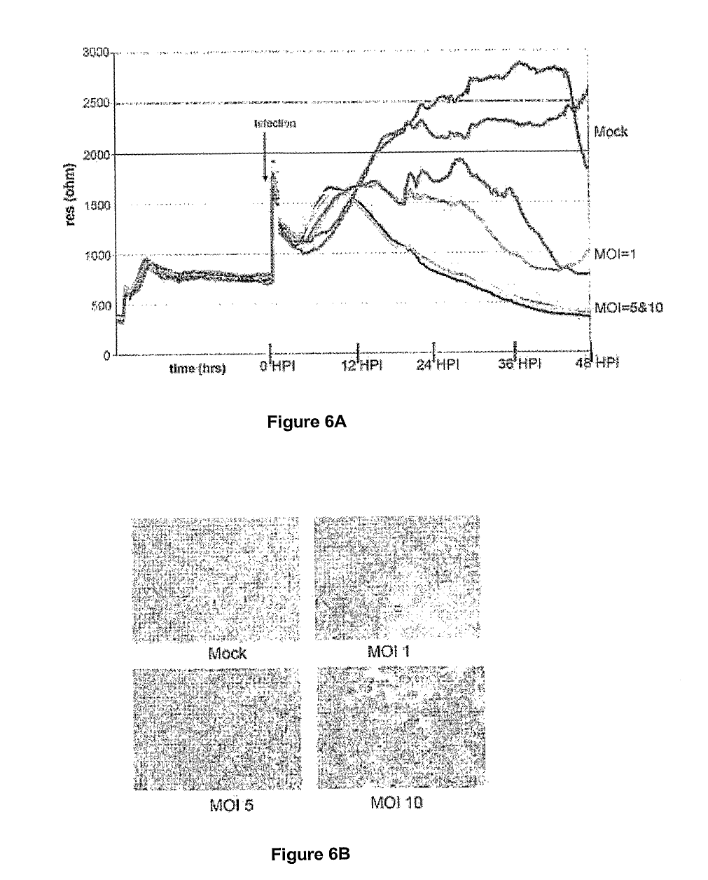

With reference to FIG. 6A, reflecting the data collected at 4,000 Hz, initially all cells, including mock controls, exhibit a spike in resistance before beginning to descend. These measurements correspond to the time directly after addition of the inoculum to the cells and therefore are attributed to the physical manipulation the cells undergo during inoculation. Approximately 3-4 hours PI, cells which receive an MOI of 5 or 10 exhibit an initial rise in resistance peaking 7-8 hours PI before falling. Although lagging slightly behind, cells which received an MOI of 1 exhibit similar readings. By 48 hours PI, the resistance of the cells infected at an MOI of 5 and 10 drop to levels approximating those prior to cell seeding. Cells infected at an MOI of 1, although not as severe, also exhibit lower resistance compared to mock controls. With reference to FIG. 6B, to confirm virus infection, the presence of cytopathic effect (CPE) is visualized and photographed 48 hours PI, illustrating the varying degree of CPE in the virus-infected cells compared to the mock control. The ECIS data is consistent with the observed CPE, which is indistinguishable in cells infected at an MOI of 5 or 10 compared to the reduced CPE seen in cells infected at an MOI of 1.

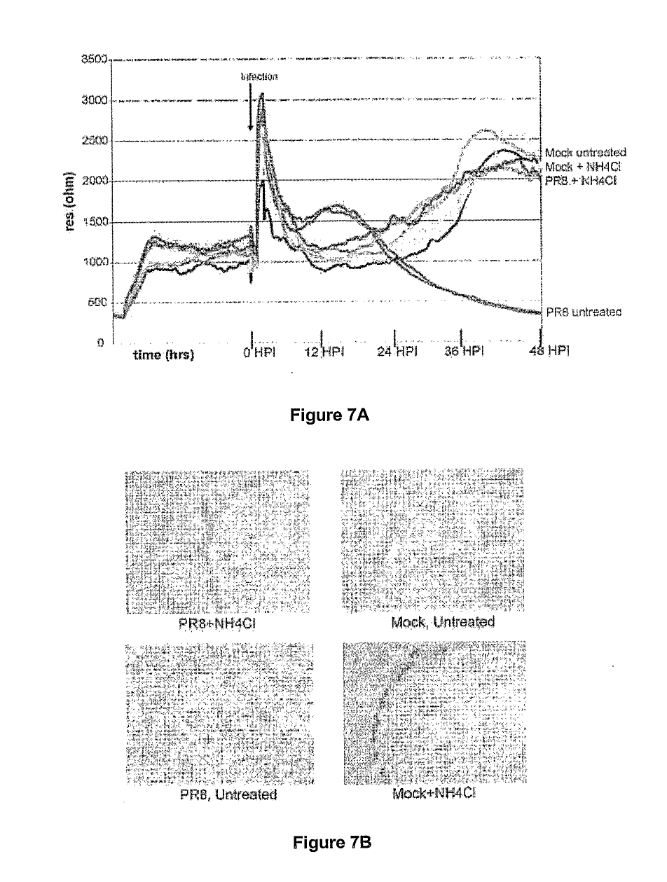

To confirm ECIS could be used for the analysis of compounds or treatment affecting virus replication, MDCK cells are seeded as before in an 8W10E multi-well culture dish. Once the resistance is stabilized, the cells are either pretreated with 20 mM NH₄C1 or left untreated in maintenance medium for 1 hour. Cells are then either inoculated with the PR8 influenza A virus at an MOI of 5 or with a mock inoculum in serum free EMEM containing 1 μg/mL TPCK treated trypsin and antibiotics. Cells pretreated with 20 mM NH₄Cl remained under NH₄Cl treatment throughout the experiment, including the inoculation period. Data are collected by ECIS as before for approximately 48 hours PI and are depicted in FIG. 7A. Pretreatment of MDCK cells with 20 mM NH₄Cl, a known virus entry inhibitor, results in similar resistance curves for both mock and influenza-inoculated cells. The only cells exhibiting the characteristic rise and subsequent fall in resistance following inoculation are the un-NH₄Cl-treated, PR8 influenza A virus-inoculated positive controls. With reference to FIG. 7B, to confirm virus infection, the presence of CPE is visualized and photographed 48 hours PI, illustrating the varying degree of CPE in the virus-infected cells, the virus-infected cells treated with NH₄Cl, and the mock controls.

Although this study involves the ECIS instrument collecting data at 3 different frequencies: 400, 4,000 and 40,000 Hz; the data collected at 4,000 Hz appears to provide more meaningful results. The general trend is the same in all three instances; however, a greater separation in the resistances measured between the variables is observed at 4,000 Hz (data not shown). Since all viral infections vary with regard to their specific kinetics of pathogenesis, it is doubtful that 4,000 Hz is optimal for all. Therefore, it is recommended that data be collected at multiple frequencies to ensure a comprehensive sampling.

Due to cytopathology in cell culture, Influenza A virus infection of MDCK cells is an appropriate test model for the use of ECIS in measuring CPE. Inhibition of CPE in influenza-infected cells through pretreatment of NH₄Cl is also observable with ECIS, illustrating its potential in screening antiviral compounds. Indeed, any viral infection resulting in CPE together with that inhibition of CPE in cell culture could theoretically be analyzed using ECIS. Additionally, in light of the observed signal resistance spike directly following manipulation of the cells at the time of inoculation and the characteristic rise and fall of resistance following influenza A virus inoculation, ECIS appears to have great sensitivity for detecting changes in cells that may not necessarily be observable under conventional microscopy. Since the number of tight junctions and the distance between the cells and the substrate to which they are attached affect the flow of current through the system, it is possible to measure changes in these two characteristics that might otherwise go unnoticed. Therefore, it may be worthwhile to use ECIS for monitoring non-cytopathic virus infections where, although no gross pathology is observed, small or subtle changes in the cell monolayer may be detected.

It will be apparent to those skilled in the art that various modifications and variations can be made in the present invention without departing from the scope or spirit of the invention. Other embodiments of the invention will also be apparent to those skilled in the art from consideration of the specification and practice of the invention disclosed herein. It is intended that the Specification and Example be considered as exemplary only, and not intended to limit the scope and spirit of the invention.

### REFERENCES

Throughout this application, various publications are referenced. All such references are incorporated herein in their entirety by reference. The following references are also incorporated herein in their entirety by reference:
Claas, E. C., de Jong, J. C., van Beek, R., Rimmelzwaan, G. F., Osterhaus, A. D., 1998. Human influenza virus A/HongKong/156/97 (H5N1) infection. Vaccine 16, 977-978.
Giaever, I., Keese, C. R., 1993. A morphological biosensor for mammalian cells. Nature 366, 591-592.
Giaever, I., Keese, C. R., 1991. Micromotion of mammalian cells measured electrically. Proc. Natl. Acad. Sci. U.S.A. 88, 7896-7900.
Giaever, I., Keese, C. R., 1986. Use of electric fields to monitor the dynamical aspect of cell behavior in tissue culture. IEEE Trans. Biomed. Eng. 33, 242-247.
Giaever, I., Keese, C. R., 1984. Monitoring fibroblasts behavior in tissue culture with an applied electric field. Proc. Natl. Acad. Sci. U.S.A. 81, 3761-3764.
Jakeman, K. J., Smith, H., Sweet, C., 1991. Influenza virus enhancement of membrane leakiness induced by staphylococcal alpha toxin, diphtheria toxin and streptolysin S. J. Gen. Virol. 72, 111-115.
Kaye, D., Pringle, C. R., 2005. Avian influenza viruses and their implication for human health. Clin. Infect. Dis. 40, 108-112.
Keese, C. R., Wegener, J., Walker, S. R., Giaever, I., 2004. Electrical wound-healing assay for cells in vitro. Proc. Natl. Acad. Sci. U.S.A. 101, 1554-1559.
Lo, C. M., Keese, C. R., Giaever, I., 1993. Monitoring motion of confluent cells in tissue culture. Exp. Cell. Res. 204, 102-109.
McCoy, M. H., and Wang, E., 2005. Use of electric cell-substrate impedance sensing as a tool for quantifying cytopathic effect in influenza A virus infected MDCK cells in real-time. J. Virological Methods 130, 157-161.
Mitra, P., Keese, C. R., Giaever, I., 1991. Electric measurements can be used to monitor the attachment and spreading of cells in tissue culture. Biotechniques 11, 504-510.
Palese, P., 2004. Influenza: old and new threats. Nat. Med. 10, S82-S87.
Smee, D. F., Morrison, A. C., Barnard, D. L., Sidwell, R. W., 2002. Comparison of colorimetric, fluorometric, and visual methods for determining anti-influenza (H1N1 and H3N2) virus activities and toxicities of compounds. J. Virol. Methods 106, 71-79.
Subbarao, K., Klimov, A., Katz, J., Regnery, H., Lim, W., Hall, H., Perdue, M., Swayne, D., Bender, C., Huang, J., Hemphill, M., Rowe, T., Shaw, J., Xu, X., Fukuda, K., Cox, N., 1998. Characterization of an avian influenza A (H5N1) virus isolated from a child with a fatal respiratory illness. Science 279, 393-396.
Tiruppathi, C., Malik, A. B., Del Vecchio, P. J., Keese, C. R., Giaever, I., 1992. Electrical method for detection of endothelial cell shape change in real time: assessment of endothelial barrier function. Proc. Natl. Acad. Sci. U.S.A. 89, 7919-7923.
Wegener, J., Keese, C. R., Giaever, I., 2000. Electric cell-substrate impedance sensing (ECIS) as a noninvasive means to monitor the kinetics of cell spreading to artificial surfaces. Exp. Cell. Res. 259, 158-166.
World Health Organization (WHO), 2006. World Health Organization: http://www.who.int/.

1. A method of measuring cytopathic effect in cells, comprising:
providing first cells in culture;
using ECIS to measure a resistance of current associated with the cells; and
quantifying a cytopathic effect associated with the cells based on the measured resistance, wherein the cytopathic effect is due to the presence or multiplication of a virus in the cells; and
collecting measurements to quantify continuously the cytopathic effect associated with the cells over a period of time.

2. The method of claim 1 and further comprising:
providing the first cells in a healthy monolayer; and
infecting the first cells with a virus.

3. The method of claim 2 and further comprising:
providing at least one additional healthy monolayer of cells;
infecting the at least one additional monolayer of cells with a concentration of the virus;
quantifying the cytopathic effect associated with each monolayer of cells based on the measured resistance; and
collecting measurements to quantify continuously the cytopathic effect associated with each monolayer cells over a period of time.

4. The method of claim 3, wherein each additional monolayer of cells that is infected with the virus is infected with a different concentration of the virus, wherein the method further comprises comparing the cytopathic effect associated with each additional monolayer of cells.

5. The method of claim 3, wherein the at least one additional monolayer of cells is of a different cell type than the first cells, wherein the method further comprises comparing the cytopathic effect associated with the different cell types.

6. The method of claim 1 and further comprising:
comparing the quantified cytopathic effect associated with the cells following infection with the quantified cytopathic effect associated with the cells prior to infection.

7. The method of claim 1 and further comprising:
comparing the quantified cytopathic effect associated with the cells to a standard curve plotting cytopathic effect as a function of resistance.

8. The method of claim 1, wherein the period of time for which measurements are collected continuously extends from a time that the first cells are provided until the occurrence of a predetermined event.

9. The method of claim 8, wherein the predetermined event is selected from: the passing of a predetermined period of time, and the measured resistance reaching a predetermined level.

10. The method of claim 1 and further comprising:
providing the first cells in a healthy monolayer;
infecting the first cells with a virus;
providing a second healthy monolayer of cells, wherein the second cells are not infected with the virus;
quantifying the cytopathic effect associated with each monolayer of cells based on the measured resistance:
collecting measurements to quantify continuously the cytopathic effect associated with each monolayer of cells over a period of time; and
comparing the cytopathic effect associated with the first cells to the cytopathic effect associated with the second cells.

11. The method of claim 10 and further comprising:
providing at least one additional healthy monolayer of cells; and
infecting the at least one additional monolayer of cells with a concentration of the virus.

12. The method of claim 11, wherein each additional monolayer of cells that is infected with the virus is infected with a different concentration of the virus, wherein the method further comprises comparing the cytopathic effect associated with each monolayer of cells.

13. The method of claim 11, wherein the at least one additional monolayer of cells is of a different cell type than the first cells, wherein the method further comprises comparing the cytopathic effect associated with the different cell types.

14. The method of claim 1 and further comprising:
providing the first cells in a healthy monolayer;
infecting the first cells with a virus;
treating the first cells with a candidate anti-viral agent; and
identifying the candidate agent as an actual antiviral agent when there is a reduction in cytopathic effect following treatment with an agent.

15. The method of claim 14 and further comprising:
providing at least one additional healthy monolayer of cells;
infecting the at least one additional monolayer of cells with a concentration of the virus;
treating the at least one additional monolayer of cells with a concentration of the candidate antiviral agent;
quantifying the cytopathic effect associated with each monolayer of cells based on the measured resistance; and
collecting measurements to quantify continuously the cytopathic effect associated with each monolayer of cells over a period of time.

16. The method of claim 15, wherein each additional monolayer of cells that is infected with the virus is infected with a different concentration of the virus, wherein the method further comprises comparing the cytopathic effect associated with each monolayer of cells.

17. The method of claim 15, wherein each additional monolayer of cells that is treated with the candidate antiviral agent is treated with a different concentration of the agent, wherein the method further comprises comparing the cytopathic effect associated with each monolayer of cells.

18. The method of claim 15, wherein the at least one additional monolayer of cells is of a different cell type than the first cells, wherein the method further comprises comparing the cytopathic effect associated with the different cell types.

19. The method of claim 1 and further comprising:
providing the first cells in a healthy monolayer;
infecting the first cells with a virus;
treating the first cells with a candidate anti-viral agent;
providing a second healthy monolayer of cells;
infecting the second cells with the virus, wherein the second cells are not treated with the candidate anti-viral agent;
quantifying the cytopathic effect associated with each monolayer of cells based on the measured resistance:
collecting measurements to quantify continuously the cytopathic effect associated with each monolayer of cells over a period of time: and
identifying the candidate agent as an actual antiviral agent if the cytopathic effect associated with the first cells is lower than the cytopathic effect associated with the second cells.

20. The method of claim 1 and further comprising:
providing a third healthy monolayer of cells, wherein the third cells are not infected with the virus.

21. The method of claim 19 and further comprising:
providing at least one additional healthy monolayer of cells;
infecting the at least one additional monolayer of cells with a concentration of the virus;
treating the at least one additional monolayer of cells with a concentration of the candidate antiviral agent;
quantifying the cytopathic effect associated with each monolayer of cells based on the measured resistance; and
collecting measurements to quantify continuously the cytopathic effect associated with each monolayer of cells over a period of time.

22. The method of claim 21, wherein each additional monolayer of cells that is infected with the virus is infected with a different concentration of the virus, wherein the method further comprises comparing the cytopathic effect associated with each monolayer of cells.

23. The method of claim 21, wherein each additional monolayer of cells that is treated with the candidate antiviral agent is treated with a different concentration of the agent, wherein the method further comprises comparing the cytopathic effect associated with monolayer of cells.

24. The method of claim 21, wherein the at least one additional monolayer of cells is of a different cell type than the first cells, wherein the method further comprises comparing the cytopathic effect associated with the different cell types.

25. The method of claim 1, wherein the first cells are infected with a virus, wherein the method further comprises treating the cells with a candidate antiviral agent; and identifying the candidate agent as an actual antiviral agent when there is a reduction in cytopathic effect following treatment with the agent.

26. The method of claim 25, wherein the first cells are infected with an unidentified virus.

27. The method of claim 1 and further comprising:
providing the first cells in a healthy monolayer;
treating the first cells with a candidate vaccine;
infecting the first cells with a virus; and
identifying the candidate vaccine as an effective vaccine if the cytopathic effect associated with the cells is maintained at or is reduced to below a predetermined level.

28. The method of claim 27 and further comprising:
providing at least one additional healthy monolayer of cells;
treating the at least one additional monolayer of cells with a concentration of the candidate vaccine; infecting the at least one additional monolayer of cells with a concentration of the virus;
quantifying the cytopathic effect associated with each monolayer of cells based on the measured resistance; and
collecting measurements to quantify continuously the cytopathic effect associated with each monolayer of cells over a period of time.

29. The method of claim 28, wherein each additional monolayer of cells that is treated with the candidate vaccine is treated with a different concentration of the vaccine, wherein the method further comprises comparing the cytopathic effect associated with with each monolayer of cells.

30. The method of claim 1 and further comprising:
providing the first cells in a healthy monolayer;
providing a second healthy monolayer of cells;
treating the first cells with a candidate vaccine;
infecting the first cells with a virus;
infecting the second cells with the virus, wherein the second cells are not treated with the candidate vaccine; quantifying the cytopathic effect associated with each cells based on the measured resistance;
collecting measurements to quantify continuously the cytopathic effect associated with each cells over a period of time; and identifying the candidate vaccine as an effective vaccine if the cytopathic effect associated with the first cells falls below and then remains lower than the cytopathic effect associated with the second cells during the period of time.

31. The method of claim 30 and further comprising:
providing at least one additional healthy monolayer of cells;
treating the at least one additional monolayer of cells with a concentration of the candidate vaccine; and
infecting the at least one additional monolayer of cells with a concentration of the virus.

32. The method of claim 31, wherein each additional monolayer of cells that is treated with the candidate vaccine is treated with a different concentration of the vaccine, wherein the method further comprises comparing the cytopathic effect associated with each additional monolayer of cells.

33. A method of measuring cytopathic effect in cells, comprising:
providing first cells in culture;
using ECIS to measure a resistance of current associated with the cells;
correlating the measured resistance to a cytopathic effect associated with the cells, wherein the cytopathic effect is due to the presence or multiplication of a virus in the cells: and
collecting measurements to quantify continuously the cytopathic effect associated with the cells over a period of time.

34. The method of claim 33 and further comprising:
providing the first cells in a healthy monolayer;
exposing the cells to a sample; and
identifying the sample as containing a virus if the cytopathic effect associated with the cells is above a predetermined level.

35. The method of claim 34, wherein the sample is a soil, a water, a food, or an animal tissue sample.

36. The method of claim 35, wherein the sample is an animal tissue sample.

37. The method of claim 33 and further comprising:
identifying the cells as being infected by a virus if the cytopathic effect associated with the cells is above a predetermined level.

38. The method of claim 37, wherein the cells are obtained from an animal tissue sample.

39. The method of claim 38, wherein the animal tissue sample is obtained from: a bird, a pig, or a cow.

40. The method of claim 38, wherein the animal tissue sample is a sample selected from an animal of a type that is used for human consumption.

41. The method of claim 37, wherein the animal tissue sample is human.

42. The method of claim 33 and further comprising:
providing the first cells in a healthy monolayer;
exposing the first cells to a concentration of a sample; and
providing the second cells in a healthy monolayer;
exposing the second cells to a concentration of a sample;
quantifying the cytopathic effect associated with each cells based on the measured resistance: and collecting measurements to quantify continuously the cytopathic effect associated with each cells over a period of time:
comparing the cytopathic effect associated with the first cells to the cytopathic effect associated with the second cells.

43. The method of claim 42, wherein the sample exposed to the first cells is different than the sample exposed to the second cells.

44. The method of claim 43, wherein the sample exposed to the cells is a soil, a water, a food, or an animal tissue sample.

45. The method of claim 43, wherein the sample exposed to the first cells is obtained from a first geographical region and the sample exposed to the second cells is obtained from a second geographical region.

46. The method of claim 45, wherein the sample exposed to the cells is a soil, a water, a food, or an animal tissue sample.

47. The method of claim 45, wherein the first cells are of a different cell type than the second cells.

48. The method of claim 42, wherein the first cells are exposed to a first concentration of a sample and the second cells are exposed to a second concentration of the same sample.

49. The method of claim 33 and further comprising:
providing second cells in culture;
quantifying the cytopathic effect associated with each cells based on the measured resistance: and
collecting measurements to quantify continuously the cytopathic effect associated with each cells over a period of time;
comparing the cytopathic effect associated with the first cells to the cytopathic effect associated with the second cells.

50. The method of claim 49, wherein the first cells are of a different cell type than the second cells.

51. The method of claim 50, wherein the first cells are obtained from a first geographical area and the second cells are obtained from a second geographical area.

52. The method of claim 49, wherein the cells are obtained from animal tissue samples.

53. The method of claim 52, wherein the animal tissue samples are obtained from: a bird, a pig, or a cow.

54. The method of claim 52, wherein the animal tissue samples are obtained from one or more animals of a type that is used for human consumption.

55. The method of claim 52, wherein at least one animal tissue sample is human.

56-60. (canceled)
